(19) United States
(12) Patent Application Publication
(10) Pub. No.: US 2022/0165390 A1
(43) Pub. Date: May 26, 2022

(54) DIGITAL THERAPEUTIC FOR TREATMENT OF PSYCHOLOGICAL ASPECTS OF AN ONCOLOGICAL CONDITION

(71) Applicant: Blue Note Therapeutics, Inc., San Francisco, CA (US)

(72) Inventors: Laura Brown CHAVAREE, San Francisco, CA (US); Mark Wesley ELFERS, Simi Valley, CA (US); Geoffrey Spencer EICH, Camarillo, CA (US); Richard Adam LIT, Malibu, CA (US); Michael John MALECKI, Westlake Village, CA (US); Michael Antone MCKINLEY, Washington, UT (US)

(21) Appl. No.: 17/100,473

(22) Filed: Nov. 20, 2020

(51) Int. Cl. G16H 20/70; G16H 10/60; G06N 3/08; G06F 3/048

(52) U.S. Cl. CPC G16H 20/70; G06F 3/048; G06N 3/08; G16H 10/60

(57) ABSTRACT

Examples described herein generally relate to a system and methods for providing a digital therapeutic for treatment of psychological aspects of an oncological condition. The digital therapeutic may generate a user interface on a user device. The user interface includes a fictional health care provider avatar and plurality of fictional patient avatars. Each fictional patient avatar associated with a distinct patient archetype. The digital therapeutic may output a prompt from the fictional health care provider avatar. The digital therapeutic may output an avatar response to the prompt from at least one selected avatar of the plurality of fictional patient avatars based on the distinct patient archetype associated with the selected avatar. The digital therapeutic may receive, from the patient, a patient response to the prompt. The digital therapeutic may select a subsequent prompt or avatar response to output based on the patient response to the prompt.

### TECHNICAL FIELD

The present disclosure relates to a digital therapeutic, and particularly to a digital therapeutic for treatment of psychological aspects of an oncological condition.

### BACKGROUND

Patients diagnosed with a serious condition such as cancer often face psychological issues. Treatment of these psychological issues via cancer-specific face-to-face behavioral interventions are associated with improved outcomes for the condition. For example, Cognitive Behavioral Stress Management (CBSM) reduced reports of thought intrusion, anxiety, and emotional distress in breast cancer patients. The beneficial effects were maintained well past the completion of adjuvant therapy. Additionally, CBSM showed significant improvement in quality of life (QoL) in prostate cancer patients. CBSM creates durable results. Patients who were treated with a CBSM face-to-face intervention showed an association with greater disease-free time and improved cancer survival. Patients treated with CBSM showed reduced serum cortisol levels and a reversal of anxiety-related up-regulation of pro-inflammatory gene expression in circulating leukocytes.

The linkage between depression and cancer mortality has been well documented, and has been shown to be independent of timing of cancer diagnosis, disease stage, type (site) of cancer, illness severity, and gender. This meta-analysis shows higher levels of depressive symptoms predicts elevated mortality. Overall, cognitive behavioral therapy (CBT) is a proven behavioral therapy which generates lasting effect, but the logistic limitations can make it impossible for patients to attend therapy. Despite various benefits, current CBT methods require patients to attend outpatient counseling sessions in a clinic setting which may not be possible for patients who are too sick to attend or who have engaged in maladaptive/avoidance to treatment behavior. These limitations are of course exacerbated during the current pandemic.

Some cancer patients may have difficulty attending group therapy sessions. For example, patients may not live close to a meeting location and travel may be difficult. Additionally, group therapy sessions may not be consistent with social distancing protocols for reducing spread of contagious diseases. Cancer patients may be reluctant to join a group therapy session during a pandemic due to the added risks. Often, available group interventions are not successful in attracting or retaining new patients due to challenges in group dynamics which can stem from differences in specific stressors (e.g., raising children), stage of life, cultural and ethnic demographics, etc.

Thus, there is a need in the art for improvements in delivery of cancer-specific face-to-face behavioral interventions.

### SUMMARY

The following presents a simplified summary of one or more implementations of the present disclosure in order to provide a basic understanding of such implementations. This summary is not an extensive overview of all contemplated implementations, and is intended to neither identify key or critical elements of all implementations nor delineate the scope of any or all implementations. Its sole purpose is to present some concepts of one or more implementations of the present disclosure in a simplified form as a prelude to the more detailed description that is presented later.

In an aspect, the disclosure provides a method of treatment of psychological aspects of an oncological condition. The method may include generating a user interface for a patient on a patient device, the user interface including a fictional health care provider avatar and plurality of fictional patient avatars, each fictional patient avatar associated with a distinct patient archetype. The method may include outputting a prompt from the fictional health care provider avatar. The method may include outputting an avatar response to the prompt from at least one selected avatar of the plurality of fictional patient avatars based on the distinct patient archetype associated with the selected avatar. The method may include receiving, from the patient, a patient response to the prompt. The method may include selecting a subsequent prompt or avatar response to output based on the patient response to the prompt. In an aspect, the method further includes receiving a selection of the plurality of fictional patient avatars from the patient.

In an aspect, selecting the subsequent prompt or avatar response includes: selecting a second health care provider prompt based on the patient response; outputting the second health care provider prompt from the fictional health care provider avatar; outputting an answer to the second health care provider prompt from a second avatar of the plurality of fictional patient avatars based on the patient archetype of the second avatar; and outputting the second health care provider prompt from the fictional health care provider avatar to the patient.

In an aspect, the method further includes determining a patient mindset of the patient.

The method may further include adding a new fictional patient avatar to the user interface, wherein a patient archetype of the new fictional patient avatar is based on the patient mindset of the patient. The patient archetype of the new fictional patient avatar may correspond to the patient mindset of the patient. The method may further include outputting, from the new fictional patient avatar, a demonstration of a skill for the patient.

In an aspect, determining the patient mindset of the patient includes providing a record of patient responses including the patient response to the prompt to a machine learning classifier, wherein the machine learning classifier is trained on a set of actual patient responses labeled with patient mindsets.

In an aspect, selecting the subsequent prompt or avatar response includes determining an emotion of the patient response based on key words included in the patient response and whether the patient response indicates distress.

In an aspect, the user interface includes a control associated with each avatar. Outputting the response to the prompt from the avatar may be in response to selection of the control for the avatar.

In an aspect, the method further includes selecting, based on a patient profile, demographic characteristics of the plurality of fictional patient avatars. A display of the fictional patient avatar may be based on the demographic characteristics. Selecting, based on a patient profile, demographic characteristics of the plurality of fictional patient avatars may include determining a cancer type of the patient and selecting at least one of the plurality of fictional patient avatars based on the cancer type. Selecting, based on a patient profile, demographic characteristics of the plurality of fictional patient avatars may include determining a mindset of the patient and selecting at least one of the plurality of fictional patient avatars based on the mindset of the patient.

In another aspect, the disclosure provides an apparatus for providing treatment of psychological aspects of an oncological condition. The apparatus may include a processor and a memory storing computer-executable instructions. The instructions, when executed by the processor, cause the processor to generate a user interface for a patient, the user interface including a fictional health care provider avatar and plurality of fictional patient avatars, each fictional patient avatar associated with a distinct patient archetype. The processor may be configured to output a prompt from the fictional health care provider avatar. The processor may be configured to output an avatar response to the prompt from at least one selected avatar of the plurality of fictional patient avatars based on the distinct patient archetype associated with the selected avatar. The processor may be configured to receive, from the patient, a patient response to the prompt. The processor may be configured to select a subsequent prompt or avatar response to output based on the patient response to the prompt.

Additional advantages and novel features relating to implementations of the present disclosure will be set forth in part in the description that follows, and in part will become more apparent to those skilled in the art upon examination of the following or upon learning by practice thereof.

### DESCRIPTION OF THE FIGURES

In the drawings:

FIG. 1 is a diagram of an example computer system for providing a digital therapeutic, in accordance with an implementation of the present disclosure.

FIG. 2 is an example message diagram illustrating example messages within a digital therapeutic, in accordance with an implementation of the present disclosure.

FIG. 3 is an example user interface for a digital therapeutic, in accordance with an implementation of the present disclosure.

FIG. 4 is an example user interface with session controls for a digital therapeutic, in accordance with an implementation of the present disclosure.

FIG. 5 is a flowchart of an example method of providing a digital therapeutic to a patient, in accordance with an implementation of the present disclosure.

FIG. 6 is a flowchart of an example method of selecting a prompt based on a patient response, in accordance with an implementation of the present disclosure.

FIG. 7 is a schematic block diagram of an example application server, in accordance with an implementation of the present disclosure.

### DETAILED DESCRIPTION

The present disclosure provides systems and methods for providing a digital therapeutic to a patient. A digital therapeutic may refer to a computer service that provides treatment for a medical condition. For example, a digital therapy may be intended to provide a patient access to therapy tools used during treatment sessions to improve recognized treatment outcomes. A digital therapeutic may also be referred to as a computerized behavioral therapy device. A computerized behavioral therapy device may be a prescription-only device intended to provide a computerized version of condition-specific behavioral therapy as an adjunct to clinician supervised outpatient treatment to patients with psychiatric conditions. A computerized behavioral therapy device for psychiatric disorders may be a Class II, prescription only device. That is, a computerized behavioral therapy device requires a prescription or a medical order from a clinician. A wellness application may be a computer service that provides general health related functionality, but may not treat a specific medical condition, and may not require a prescription.

In an aspect, the digital therapeutic of the present disclosure provides the experience, and sociocognitive benefits, of a group behavioral therapy session through fictional digital avatars. The digital therapeutic may present a digital group behavioral therapy session led by a fictional health care provider avatar. The fictional digital patient avatars may each be associated with a distinct patient archetype and/or a patient mindset. A patient archetype may refer to a classification of patient characteristics. Patient archetypes may be derived from research with healthcare providers and listings and categorizations of important patient dialogue, life examples, and coping challenges that enable valuable group discussion. Example patient archetypes include a patient struggling to confront existential issues, a reserved or quiet patient, a Super Coper patient that is adept at coping with cancer, a defensive and controlling patient, an older, skeptical patient, a patient dealing with recurrence or metastasis, and a resistant patient. Patient archetypes may provide key points in intervention content. In an aspect, patient archetypes may serve as a framework for generating content for a fictional patient avatar. The patient archetype may determine a set of available responses.

A patient mindset may refer to another classification of patient characteristics. Patient mindsets may be derived from patient research and affinity exercises. The patient mindsets may be tested to determine reproducibility and repeatability. For example, in an aspect, a cancer patient may be classified into one of five mindsets. A first mindset may be referred to as a step-by-step mindset. A patient with a step-by-step mindset may focus on short term goals within the cancer journey and may need to feel that they are working toward a clear goal. A patient with a step-by-step mindset may have a small circle of support and may want a deeper connection with a support system. A second mindset may be referred to as a hold-it-in mindset. A patient with a hold-it-in mindset may be complacent with regards to the cancer journey. The patient with the hold-it-in mindset may process challenges internally, but may be open to receiving help out of a feeling of obligation. The patient with the hold-it-in mindset may be selective about what is shared and with whom it is shared. A third mindset may be referred to as a wholly engaged mindset. A patient with a wholly engaged mindset may be actively involved, for example, by doing research and sharing with others. The patient with the wholly engaged mindset may have different types of support and may be receptive to new concepts. A fourth mindset may be referred to as a get-up, get-on mindset. A patient with the get-up, get-on mindset may not dwell on the future. The patient with the get-up, get-on mindset may have a single source of emotional support and care such as a spouse. The patient with the get-up, get-on mindset may compartmentalize cancer, may not be interested in sharing, and may not be willing to join group therapy. The patient with the get-up, get-on mindset may be at high risk of depressive effects and may benefit from therapy. A fifth mindset may be referred to as a push-through mindset. A patient with a push-through mindset may not set goals but tries to move forward. The patient with the push-through mindset may lack an existing support network, but may enjoy high-touch interactions with the community. The patient with the push-through mindset may keep busy to redirect anxiety.

Each patient mindset may be associated with one or more compatible patient archetypes. Each fictional patient avatar may be assigned a patient mindset that is compatible with the patient archetype. The patient mindset may further inform content for a fictional patient avatar with the corresponding patient archetype. For example, a patient archetype may be associated with two possible answers to a prompt depending on the patient mindset of the fictional patient avatar. Additionally, a mindset of a real patient may be identified based on responses to interview, affinity exercises, and group therapy sessions. Generally, interaction between a real patient and a fictional patient avatar may be most beneficial when the mindset of the real patient corresponds to an archetype of the fictional patient avatar. For example, the fictional patient avatar may demonstrate skills that are consistent with the mindset of the real patient. Accordingly, the real patient may emulate the skill demonstrated by the fictional patient avatar. In an aspect, the digital therapeutic may alter the digital experience of the real patient to affect the user experience based on responses from the patient that identify a mindset of the patient. For instance, the digital therapeutic may add new fictional avatars or change a patient mindset of an existing fictional avatar based on the mindset of the patient.

The digital therapeutic may provide a plurality of group behavioral therapy sessions structured according to a therapeutic flow defined by a physician or other healthcare expert. Each group behavioral therapy session may be associated with one or more pre-requisites (e.g., onboarding procedure or completed sessions), a goal, and a completion requirement. Each group behavioral therapy session may include a series of prompts provided by the fictional health care provider, responses from fictional patient avatars, and response opportunities for the patient-user. The digital therapeutic may include a library of avatar responses associated with patient archetype, patient mindset, and demographics. The digital therapeutic may select avatar responses from the library based on the goal for the session, the archetype or mindset of the fictional avatar, and a profile of the patient including analysis of responses from the patient. Accordingly, the experience of the patient using the digital therapeutic may adapt to the patient based on the responses of the patient.

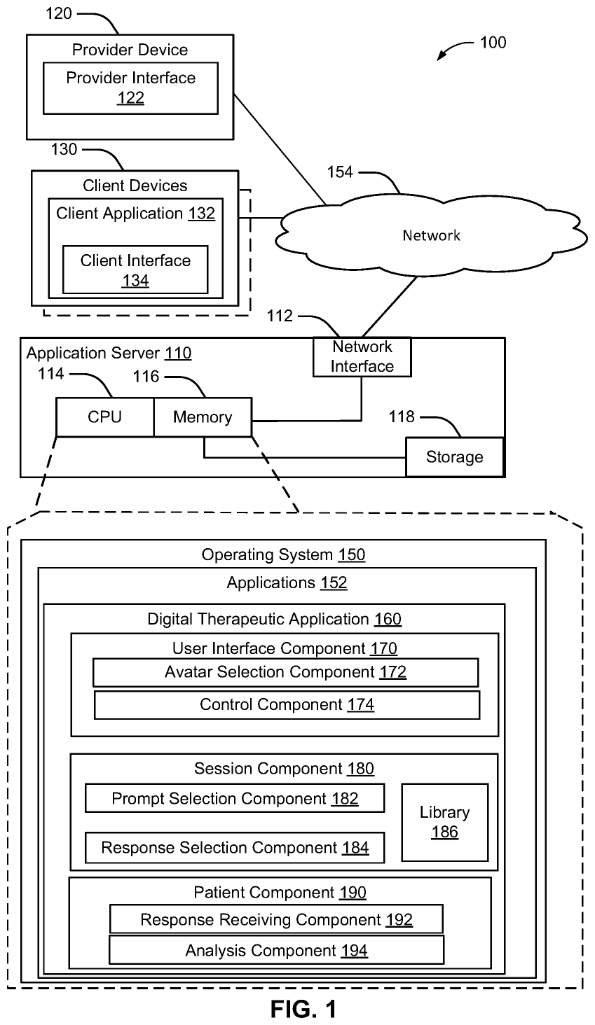

Referring now to FIG. 1, an example digital therapeutic system 100 includes a central application server 110 and a plurality of user devices including at least one provider device 120, and a plurality of client devices 130. The application server 110 may be, for example, any mobile or fixed computer device including but not limited to a computer server, desktop or laptop or tablet computer, a cellular telephone, a personal digital assistant (PDA), a handheld device, any other computer device having wired and/or wireless connection capability with one or more other devices, or any other type of computerized device capable of processing communications related data. In an aspect, the application server 110 may be implemented as one or more virtual machines hosted by a web services provider.

In an aspect, the digital therapeutic system 100 may include a digital therapeutic application 160 executed by the application server 110 that the digital therapeutic system 100 operates to provide a patient with the digital therapeutic via one of the client devices 130. In an aspect, the digital therapeutic application 160 may employ a client/server architecture with a client application 132 at the client device 130. Although various functions and data are described herein as being located at either the client application 132 or the digital therapeutic application 160, it should be appreciated that such functions and data may be distributed between the client application 132 and the digital therapeutic application 160, for example, based on computing resources such as storage capacity and network bandwidth. For example, the digital therapeutic application 160 may store a complete library 186 of fictional patient and provider avatars, prompts and responses, which may be streamed to the client application 132, but the client application 132 may download a portion of the library 186 based on scheduled sessions and/or selected avatars for offline use.

The digital therapeutic application 160 may include user interface component 170 that controls the client interface 134 of the client application 132. In particular, the user interface component 170 may include an avatar selection component 172 and a control component 174. The avatar selection component 172 may select an avatar based on a patient profile and/or input from the patient. In an aspect, the avatar selection component 172 may present a user interface that allows the patient to select a group of fictional patient avatars from a library. The user interface may provide a profile of each fictional patient avatar including one or more of a name, age, sex, medical condition, race, ethnicity, religion, or family status. The user interface may allow the patient to filter the fictional patient avatars based on desired characteristics. In some implementations, the avatar selection component 172 may suggest or select fictional patient avatars for the patient based on a patient profile. For example, the avatar selection component 172 may select fictional patient avatars with characteristics matching the patient. In particular, the avatar selection component 172 may select fictional patient avatars that may provide a useful perspective for the patient within the context of that patient's specific disease, disease progression, or demographics. For instance, the avatar selection component 172 may select fictional patient avatars that represent various stages of the medical condition.

In an aspect, each fictional patient avatar is associated with a patient archetype. The patient archetype may be a hidden characteristic that is not directly presented to the patient. The patient archetype may be the primary factor in selecting a patient avatar response to be presented by the fictional patient avatar. The avatar selection component 172 may select the patient archetype for each fictional patient avatar based on the patient profile or patient responses to the other avatars regardless of other patient characteristics.

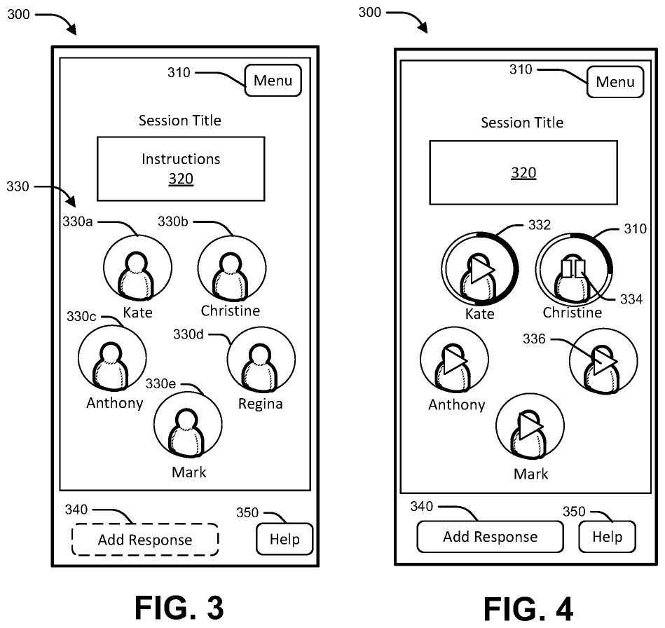

The control component 174 may provide patient controls over a session. The control component 174 may allow the patient to select a fictional patient avatar and play or pause a response from the fictional patient avatar. The control component 174 may also include controls to start a prompt from the fictional health care provider and to start recording a patient response. The control component 174 may provide the user interfaces illustrated in FIGS. 3 and 4. The control component 174 may receive input via the user interface indicating a selected control and execute a corresponding function.

The digital therapeutic application 160 may include a session component 180 that controls content of a session for a patient. The session component 180 may include a prompt selection component 182, a response selection component 184, and a library 186. A session may be structured around a defined series of prompts. The series of prompts for a session may be based on a behavioral therapy intervention. The prompt selection component 182 may select the series of prompts for the session. The prompt selection component 182 may determine when to present each prompt based on a condition, which may include a number of output fictional patient avatar responses and/or an input patient response. In an implementation, one or more of the prompts may be associated with a secondary prompt. The secondary prompt may be selected for inclusion in the session based on a patient response. For example, the secondary prompt may be based on an emotion indicated by the patient response. For instance, the secondary prompt may ask a fictional patient avatar how the avatar has handled the emotion indicated by the patient response, then ask the patient whether the fictional patient avatar's response would be helpful. U.S. patent application Ser. No. 17/078,987 discloses techniques for detecting emotion and is incorporated herein by reference.

The response selection component 184 may select fictional patient avatar responses to a prompt. In an aspect, the response selection component 184 may select a response for each fictional avatar based on the patient archetype of the avatar. In some implementations, the response may be customized based on demographic characteristics of the avatar. For example, names or pronouns may be inserted into a script that includes a substantive response to the prompt. The avatar responses may be output as an audio output, as a text display, or as both. In an implementation, the avatar responses may be stored as text and the audio output may be generated via a text to speech function. A different voice for the text to speech function may be associated with each avatar.

The digital therapeutic application 160 may include a patient component 190. The patient component 190 may include a response receiving component 192 and an analysis component 194. The response receiving component 192 may receive a patient response from the client device 130. For example, the patient response may be a video file, an audio file, or a text file. The patient response may be associated with the prompt that resulted in the response.

The analysis component 194 may analyze the response to determine content and context. In an aspect, the analysis component 194 may convert an audio file to a text file. The analysis component 194 may search the text file for keywords based on the associated prompt. For example, the keywords may include words that are associated with particular emotions.

In an aspect, the analysis component 194 may include a machine-learning classifier trained to classify one or more patient responses into a limited number of patient mindsets (e.g., 5). The machine learning classifier may be trained using supervised learning techniques. A training set may include transcripts of patient interviews where the patient answered prompts. The transcripts may be manually classified by a health care provider and labelled with the classification. The machine-learning classifier may include a support vector machine (SVM) trained on the training set to classify a transcript of one or more patient responses into one of the limited number of patient mindsets. The analysis component 194 may provide a classification of a patient to the avatar selection component 172, the prompt selection component 182 and/or the response selection component 184.

The application server 110 may include a central processing unit (CPU) 114 that executes instructions stored in memory 116. For example, the CPU 114 may execute an operating system 150 and one or more applications 152, which may include the digital therapeutic application 160. The application server 110 may also include a network interface 112 for communication with external devices via a network 154. For example, the application server 110 may communicate with a plurality of user devices including the provider device 120 and client devices 130.

Memory 116 may be configured for storing data and/or computer-executable instructions defining and/or associated with an operating system 150 and/or application 152, and CPU 114 may execute operating system 150 and/or applications 152. Memory 116 may represent one or more hardware memory devices accessible to application server 110. An example of memory 116 can include, but is not limited to, a type of memory usable by a computer, such as random access memory (RAM), read only memory (ROM), tapes, magnetic discs, optical discs, volatile memory, non-volatile memory, and any combination thereof. Memory 116 may store local versions of applications being executed by CPU 114. In an aspect, the memory 116 may include or communicate with a storage device 118, which may be a non-volatile memory.

The CPU 114 may include one or more processors for executing instructions. An example of CPU 114 can include, but is not limited to, any processor specially programmed as described herein, including a controller, microcontroller, application specific integrated circuit (ASIC), field programmable gate array (FPGA), system on chip (SoC), or other programmable logic or state machine. The CPU 114 may include other processing components such as an arithmetic logic unit (ALU), registers, and a control unit. The CPU 114 may include multiple cores and may be able to process different sets of instructions and/or data concurrently using the multiple cores to execute multiple threads. In an aspect, a graphics processing unit (GPU) may perform some operations of the CPU 114.

The operating system 150 may include instructions (such as applications 152) stored in memory 116 and executable by the CPU 114. The applications 152 may include a digital therapeutic application 160 configured to communicate with user devices via a respective interface (e.g., provider interface 122 or client interface 134). The digital therapeutic application 160 may provide the provider interface 122 that may be in communication with or otherwise operate in conjunction with a provider device 120. The provider interface 122 may be a graphical user interface (GUI) with which an end user may interact. For example, the provider interface 122 may be a web-page that is accessed through a browser application executed on the provider device 120. By loading the web-page, the browser application may effectively operate as a user interface for an application executed on the application server 110 (e.g., in the case of a web server). As another example, the provider interface 122 may be an application or operating system that runs on the provider device 120.

The digital therapeutic application 160 may also provide the client interface 134 that may be in communication with or otherwise operate in conjunction with a client device 130. The client interface 134 may be any user interface with which an end user may interact. For example, the client interface 134 may be a web-page that is accessed through a browser application (client) executed on the client device 130. By loading the web-page, the browser application may effectively operate as a user interface for an application executed on the application server 110 (e.g., in the case of a web server). Such an aspect may allow various types of user devices to serve as a client device 130 and participate in a digital therapeutic. For example, a communication session may include different types of client devices 130 such as desktop computers, laptop computers, tablets, and smart phones. In an aspect, the client interface 134 may be provided by a client application 132, which may be a stand-alone application installed on the client device 130.

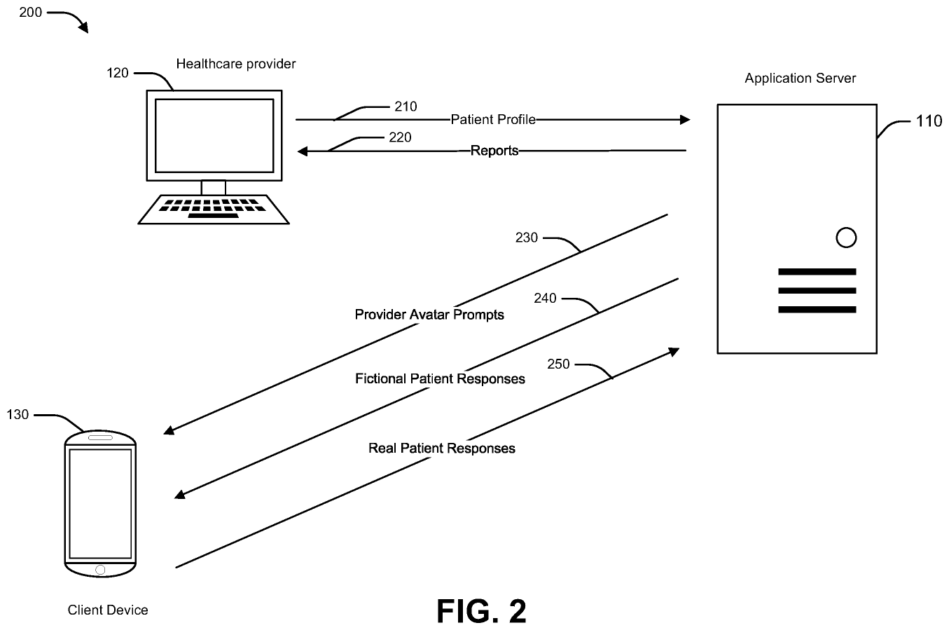

FIG. 2 is an example message diagram 200 illustrating example messages for providing a digital therapeutic. The provider device 120 may initiate the digital therapeutic via an onboarding process. In an aspect, the digital therapeutic may be prescribed for the patient according to a prescription standard such as a NCPDP standard using a NDC-like code for the digital therapeutic.

The provider device 120 may transmit a patient profile 210 to the application server 110. The patient profile 210 may include patient identifying information, patient demographic information, and patient medical information. In some implementations, the patient medical information may include a prescribed therapeutic course including one or more behavioral therapy sessions. The application server 110 may receive the patient profile 210 and associate the patient profile 210 with a patient account and a client device 130.

The application server 110 may transmit reports 220 to the provider device 120. The reports 220 may indicate progress of the patient regarding the therapeutic course. For example, the reports 220 may include a number of sessions completed. In some implementations, the reports 220 may include patient responses 250. Accordingly, a health care provider at the provider device 120 may monitor the patient. In an aspect, the provider device 120 may send an updated patient profile 210 that changes the therapeutic course for the patient.

The application server 110 may transmit provider avatar prompts 230 to the client device 130. The provider avatar prompts 230 may cause the client device 130 to output a prompt from the fictional health care provider avatar. For instance, the prompt may be a textual or audio request or question.

The application server 110 may transmit fictional patient responses 240 to the client device 130. The fiction patient responses 240 may cause the client device 130 to output, from at least one patient avatar, a response to the prompt based on the distinct patient archetype associated with the fictional patient avatar. In some implementations, the application server 110 may transmit a textual patient avatar response or stream an audio patient avatar response from the library 186. In other implementations, the user device 130 may store a copy of at least a portion of the library 186 and output the locally stored response.

The client device 130 may transmit real patient responses 250 to the application server 110. The real patient responses 250 may include textual, audio, or video responses. For instance, the client device 130 may accept input of the real patient response 250 via a keyboard, microphone, or camera of the client device 130.

FIG. 3 is an example user interface 300 for presenting a virtual group therapy session. The user interface 300 may include a menu button 310, instructions 320, and a plurality of avatars 330. The user interface 300 may further include an add response button 340 and a help button 350. The menu button 310 may present a list of control options such as selecting a session, changing fictional patient avatars, and exiting the application. The instructions 320 may provide a user with instructions for operating the user interface 300. For example, the instructions 320 may instruct the user to tap or click on one of the fictional patient avatars 330. The plurality of avatars 330 may represent participants in the virtual group therapy session. Each avatar 330 may be associated with an image and a name. In an aspect, one of the avatars may represent a health care provider. For example, avatar 330a may be the health care provider avatar and the instructions 320 may instruct the user to first select the avatar 330 to start the session. The other avatars 330 (e.g., 330b, 330c, 330d, 330e) may be virtual patient avatars. The add response button 340 may activate a keyboard, microphone, and/or camera for the user to record a response to a prompt. In an aspect, the add response button 340 may be disabled until the user fulfills a condition. For example, the add response button 340 may be enabled in response to the user playing a minimum number (e.g., 2) of the patient avatar responses. The help button 350 may provide further explanation of the instructions 320.

FIG. 4 is an example of the user interface 300 during a virtual therapy session. The user interface 300 still includes the menu button 310, instructions 320, avatars 330, add response button 340, and help button 350. The avatars 330 may include controls for controlling avatar responses. For example, an avatar that has been selected may include a progress indicator 332 showing progress on a current prompt or response. For example, as illustrated, the avatar 330a may have completed a first part of a prompt, and the avatar 330b may be currently playing a response. The progress indicator 332 may indicate approximately half completion for the avatar 330a and a quarter completion for the avatar 330b. The user may pause an active avatar by selecting a pause control 334. The user may switch to a different avatar by selecting a play control 336. The play control 336 may pause the current avatar and start output from the avatar 330 associated with the play control 336. In some implementations, the play control 336 may be disabled until the current avatar reaches a specified point. For instance, the output from a selected avatar may provide a response to the previous output. The add response button 340 may be enabled once the condition has been satisfied. In response to the user selecting the add response button 340, the user interface 300 may present a choice of text, audio, or video input and activate the corresponding input device in response to a selection from the user.

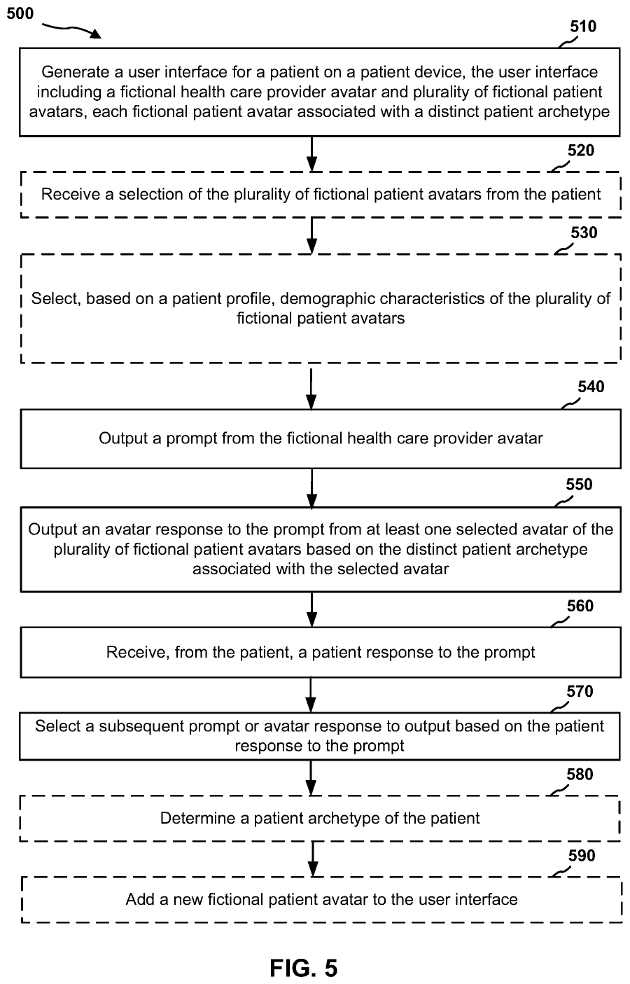

FIG. 5 is a flowchart of an example method 500 of providing a virtual group therapy session. The method 500 may be performed by the application server 110 in communication with a client device 130. The application server 110 may include the CPU 114 executing the digital therapeutic application 160. The client device 130 may similarly include a CPU executing the client application 132.

At block 510, the method 500 may include generating a user interface for a patient on a patient device, the user interface including a fictional health care provider avatar and plurality of fictional patient avatars, each fictional patient avatar associated with a distinct patient archetype. For example, the application server 110 (e.g., user interface component 170) and/or the client application 132 may generate the user interface 300. For example, the interface component 170 may send commands to the client application 132, and the client application 132 may generate the user interface 300 on the client device 130. The user interface 300 may include a fictional health care provider avatar (e.g., avatar 330a) and a plurality of fictional patient avatars (e.g., avatars 330b, 330c, 330d, 330e).

At block 520, the method 500 may optionally receiving a selection of the plurality of fictional patient avatars from the patient. For example, the application server 110 (e.g., the avatar selection component 172) may receive the selection of the plurality of fictional patient avatars from the patient.

At block 530, the method 500 may optionally include selecting, based on a patient profile, demographic characteristics of the plurality of fictional patient avatars. For example, the application server 110 may receive the patient profile 210. The avatar selection component 172 may select the demographic characteristics of the plurality of fictional patient avatars. For example, the patient profile may include a cancer type of the patient. The avatar selection component 172 may select fictional patient avatars that match the cancer type of the patient. For instance, for a cancer that primarily affects males (e.g., testicular cancer or prostate cancer), the avatar selection component 172 may select male fictional avatars. As another example, where the patient profile indicates a psychological profile or mindset of the patient, the avatar selection component 172 may select a mindset for one or more of the fictional avatars that matches the mindset of the patient.

At block 540, the method 500 may include outputting a prompt from the fictional health care provider avatar. For example, the prompt selection component 182 may select the prompt and the control component 174 may control the output of the prompt on the client device 130 via the client application 132. In some implementations, the control component 174 may highlight or otherwise indicate an active speaker. The control component 174 may play an audio or video file including the prompt. In some implementations, the control component 174 may present a text prompt either alone or concurrently with the audio/video prompt (e.g., as subtitles).

At block 550, the method 500 may include outputting an avatar response to the prompt from at least one selected avatar of the plurality of fictional patient avatars based on the distinct patient archetype associated with the selected avatar. In an aspect, the at least one selected avatar may be selected by a user via the user interface 300, for example, by selecting the avatar 330 or a play control 336 associated with the avatar 330.

At block 560, the method 500 may include receiving, from the patient, a patient response to the prompt. In an aspect, for example, the application server 110 (e.g., the response receiving component 192) may receive the patient response to the prompt. In an aspect, the response receiving component 192 may receive a text file, audio file, or video file generated by the client application 132 in response to the patient selecting the add response button 340.

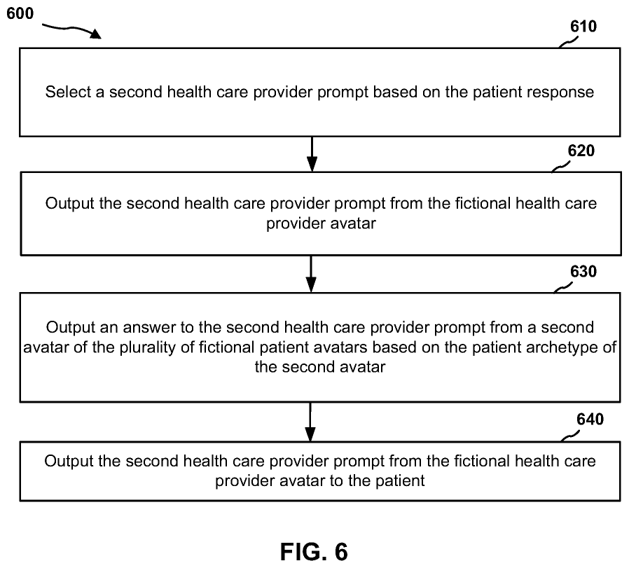

At block 570, the method 500 may include selecting a subsequent prompt or avatar response to output based on the patient response to the prompt. For example, the session component 180 may select the subsequent prompt or avatar response. In an aspect, the subsequent prompt or avatar response may be based on the patient response being submitted. For example, the subsequent prompt or avatar response may be a next prompt or avatar response in a session flow. In some implementations, the subsequent prompt or avatar response may be based on content of the patient response. For example, FIG. 6 illustrates an example method of selecting the subsequent prompt or avatar response based on a patient response.

At block 580, the method 500 may include determining a patient archetype of the patient. For example, the analysis component 194 may determine the patient archetype of the patient. In an aspect, determining the patient archetype of the patient may include providing a record of patient responses including the patient response to the prompt to a machine learning classifier. The machine learning classifier may be trained on a set of actual patient responses labeled with patient archetypes to classify the record of patient responses into the patient archetype.

At block 590, the method 500 may include adding a new fictional patient avatar to the user interface. For example, the avatar selection component 172 may add the new fictional patient avatar to the client interface 134. The new fictional patient avatar may be associated with a patient archetype that matches the patient archetype of the patient. The new fictional patient avatar may demonstrate a skill that the patient may use. For example, the new fictional patient avatar may demonstrate an interaction with a health care provider that is consistent with the patient archetype of the patient.

FIG. 6 is a flowchart of an example method 600 of selecting a subsequent prompt or avatar response in response to a patient response. The method 600 may be performed by the application server 110 in communication with a client device 130. The application server 110 may include the CPU 114 executing the digital therapeutic application 160. The client device 130 may similarly include a CPU executing the client application 132. The method 600 may correspond to block 570 of method 500.

At block 610, the method 600 may include selecting a second health care provider prompt based on the patient response. For example, the prompt selection component 182 may select the second health care provider prompt from a set of prompts configured for the session. The selection of the second health care provider prompt may be based on content of the patient response. For example, the analysis component 194 may determine an emotion of the patient response based on key words included in the patient response and whether the patient response indicates distress. The prompt selection component 182 may then select the second health care provider prompt based on the determined emotion. In some implementations, a first health care provider prompt may be associated with a plurality of second health care provider prompts, each of which corresponds to an emotion.

At block 620, the method 600 may include outputting the second health care provider prompt from the fictional health care provider avatar. For example, the control component 174 may output the second health care provider prompt via the client interface 134.

At block 630, the method 600 may include outputting an answer to the second health care provider prompt from a second avatar of the plurality of fictional patient avatars based on the patient archetype of the second avatar. For example, the response selection component 184 may select the answer to the second health care provider prompt. In an aspect, the second avatar may be selected based on a mindset of the patient. For instance, the second avatar may be selected to model how a person with a similar mental state may positively respond to the emotion experienced by the patient as indicated in the patient response.

At block 640, the method 600 may include outputting the second health care provider prompt from the fictional health care provider avatar to the patient. For example, the control component 174 may output the second health care provider prompt via the client interface 134. The second health care provider prompt may be addressed to the patient and may instruct the patient to record a second patient response. Accordingly, the patient may experience the output from the second avatar prior to recording a response to the second prompt.

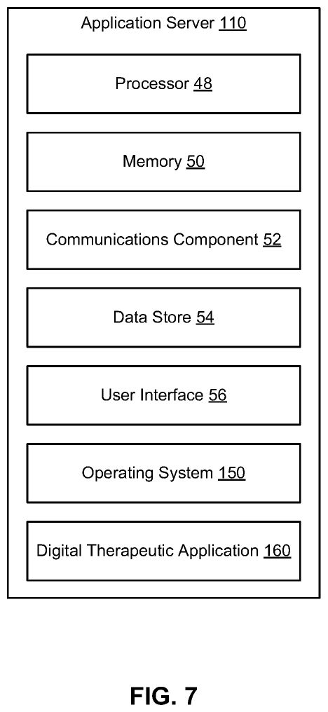

Referring now to FIG. 7, illustrated is an example application server 110 in accordance with an aspect, including additional component details as compared to FIG. 1. In one example, application server 110 may include processor 48 for carrying out processing functions associated with one or more of components and functions described herein. Processor 48 can include a single or multiple set of processors or multi-core processors. Moreover, processor 48 can be implemented as an integrated processing system and/or a distributed processing system. In an aspect, for example, processor 48 may include CPU 114.

In an example, application server 110 may include memory 50 for storing instructions executable by the processor 48 for carrying out the functions described herein. In an aspect, for example, memory 50 may include memory 116. The memory 50 may include instructions for executing the digital therapeutic application 160.

Further, application server 110 may include a communications component 52 that provides for establishing and maintaining communications with one or more parties utilizing hardware, software, and services as described herein. Communications component 52 may carry communications between components on c application server 110, as well as between application server 110 and external devices, such as devices located across a communications network 154 and/or devices serially or locally connected to application server 110. For example, communications component 52 may include one or more buses, and may further include transmit chain components and receive chain components associated with a transmitter and receiver, respectively, operable for interfacing with external devices.

Additionally, application server 110 may include a data store 54, which can be any suitable combination of hardware and/or software, that provides for mass storage of information, databases, and programs employed in connection with aspects described herein. For example, data store 54 may be a data repository for operating system 150 and/or applications 152. The data store may include memory 116 and/or storage device 118.

Application server 110 may also include a user interface component 56 operable to receive inputs from a user of application server 110 and further operable to generate outputs for presentation to the user. User interface component 56 may include one or more input devices, including but not limited to a keyboard, a number pad, a mouse, a touch-sensitive display, a digitizer, a navigation key, a function key, a microphone, a voice recognition component, any other mechanism capable of receiving an input from a user, or any combination thereof. Further, user interface component 56 may include one or more output devices, including but not limited to a display, a speaker, a haptic feedback mechanism, a printer, any other mechanism capable of presenting an output to a user, or any combination thereof

In an aspect, user interface component 56 may transmit and/or receive messages corresponding to the operation of operating system 150 and/or applications 152. In addition, processor 48 may execute operating system 150 and/or applications 152, and memory 50 or data store 54 may store them.

As used in this application, the terms “component,” “system” and the like are intended to include a computer-related entity, such as but not limited to hardware, firmware, a combination of hardware and software, software, or software in execution. For example, a component may be, but is not limited to being, a process running on a processor, a processor, an object, an executable, a thread of execution, a program, and/or a computer. By way of illustration, both an application running on a computer device and the computer device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Various aspects or features may have been presented in terms of systems that may include a number of devices, components, modules, and the like. A person skilled in the art should understand and appreciate that the various systems may include additional devices, components, modules, etc. and/or may not include all of the devices, components, modules etc. discussed in connection with the figures. A combination of these approaches may also be used.

The various illustrative logics, logical blocks, and actions of methods described in connection with the embodiments disclosed herein may be implemented or performed with a specially-programmed one of a general purpose processor, a GPU, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computer devices, e.g., a combination of a DSP and a microprocessor, a plurality of microprocessors, one or more microprocessors in conjunction with a DSP core, or any other such configuration. Additionally, at least one processor may comprise one or more components operable to perform one or more of the steps and/or actions described above.

Further, the steps and/or actions of a method or procedure described in connection with the aspects disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of storage medium known in the art. An exemplary storage medium may be coupled to the processor, such that the processor can read information from, and write information to, the storage medium. In the alternative, the storage medium may be integral to the processor. Further, in some aspects, the processor and the storage medium may reside in an ASIC. Additionally, the ASIC may reside in a user terminal. In the alternative, the processor and the storage medium may reside as discrete components in a user terminal. Additionally, in some aspects, the steps and/or actions of a method or procedure may reside as one or any combination or set of codes and/or instructions on a machine readable medium and/or computer readable medium, which may be incorporated into a computer program product.

In one or more aspects, the functions described may be implemented in hardware, software, firmware, or any combination thereof. If implemented in software, the functions may be stored or transmitted as one or more instructions or code on a computer-readable medium. Computer-readable media includes both computer storage media and communication media including any medium that facilitates transfer of a computer program from one place to another. A storage medium may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

While aspects of the present disclosure have been described in connection with examples thereof, it will be understood by those skilled in the art that variations and modifications of the aspects described above may be made without departing from the scope hereof. Other aspects will be apparent to those skilled in the art from a consideration of the specification or from a practice in accordance with examples disclosed herein.

What is claimed is:

1. A method of treatment of psychological aspects of an oncological condition, comprising:
generating a user interface for a patient on a patient device, the user interface including a fictional health care provider avatar and plurality of fictional patient avatars, each fictional patient avatar associated with a distinct patient archetype outputting a prompt from the fictional health care provider avatar;
outputting an avatar response to the prompt from at least one selected avatar of the plurality of fictional patient avatars based on the distinct patient archetype associated with the selected avatar;
receiving, from the patient, a patient response to the prompt; and
selecting a subsequent prompt or avatar response to output based on the patient response to the prompt.

2. The method of claim 1, further comprising:
receiving a selection of the plurality of fictional patient avatars from the patient.

3. The method of claim 1, wherein selecting the subsequent prompt or avatar response comprises:
selecting a second health care provider prompt based on the patient response;
outputting the second health care provider prompt from the fictional health care provider avatar;
outputting an answer to the second health care provider prompt from a second avatar of the plurality of fictional patient avatars based on the patient archetype of the second avatar; and
outputting the second health care provider prompt from the fictional health care provider avatar to the patient.

4. The method of claim 1, further comprising determining a patient mindset of the patient.

5. The method of claim 4, further comprising adding a new fictional patient avatar to the user interface, wherein a patient archetype of the new fictional patient avatar is based on the patient mindset of the patient.

6. The method of claim 5, wherein the patient archetype of the new fictional patient avatar corresponds to the patient mindset of the patient, further comprising outputting, from the new fictional patient avatar, a demonstration of a skill for the patient.

7. The method of claim 4, wherein determining the patient mindset of the patient comprises providing a record of patient responses including the patient response to the prompt to a machine learning classifier, wherein the machine learning classifier is trained on a set of actual patient responses labeled with patient mindsets.

8. The method of claim 1, wherein selecting the subsequent prompt or avatar response comprises:
determining an emotion of the patient response based on key words included in the patient response and whether the patient response indicates distress.

9. The method of claim 1, wherein the user interface includes a control associated with each avatar, wherein the outputting the response to the prompt from the avatar is in response to selection of the control for the avatar.

10. The method of claim 1, further comprising:
selecting, based on a patient profile, demographic characteristics of the plurality of fictional patient avatars, wherein a display of the fictional patient avatar is based on the demographic characteristics.

11. The method of claim 10, wherein selecting, based on a patient profile, demographic characteristics of the plurality of fictional patient avatars comprises determining a cancer type of the patient and selecting at least one of the plurality of fictional patient avatars based on the cancer type.

12. The method of claim 10, wherein selecting, based on a patient profile, demographic characteristics of the plurality of fictional patient avatars comprises determining a mindset of the patient and selecting at least one of the plurality of fictional patient avatars based on the mindset of the patient.

13. An apparatus for providing treatment of psychological aspects of an oncological condition, comprising:
a processor; and
a memory storing computer-executable instructions that when executed by the processor, cause the processor to:
generate a user interface for a patient, the user interface including a fictional health care provider avatar and plurality of fictional patient avatars, each fictional patient avatar associated with a distinct patient archetype;
output a prompt from the fictional health care provider avatar;
output an avatar response to the prompt from at least one selected avatar of the plurality of fictional patient avatars based on the distinct patient archetype associated with the selected avatar;
receive, from the patient, a patient response to the prompt; and
select a subsequent prompt or avatar response to output based on the patient response to the prompt.

14. The apparatus of claim 13, wherein the processor is configured to receive a selection of the plurality of fictional patient avatars from the patient.

15. The apparatus of claim 13, wherein the processor is configured to:
select a second health care provider prompt based on the patient response;
output the second health care provider prompt from the fictional health care provider avatar;
output an answer to the second health care provider prompt from a second avatar of the plurality of fictional patient avatars based on the patient archetype of the second avatar; and
output the second health care provider prompt from the fictional health care provider avatar to the patient.

16. The apparatus of claim 13, wherein the processor is configured to determine a patient archetype of the patient.

17. The apparatus of claim 16, wherein the processor is configured to add a new fictional patient avatar to the user interface, wherein a patient archetype of the new fictional patient avatar is based on the patient archetype of the patient.

18. The apparatus of claim 17, wherein the patient archetype of the new fictional patient avatar is the same as the patient archetype of the patient, wherein the processor is configured to output, from the new fictional patient avatar, a demonstration of a skill for the patient.

19. The apparatus of claim 18, wherein the processor is configured to provide a record of patient responses including the patient response to the prompt to a machine learning classifier, wherein the machine learning classifier is trained on a set of actual patient responses labeled with patient mindsets.

20. The apparatus of claim 13, wherein the processor is configured to determine an emotion of the patient response based on key words included in the patient response and whether the patient response indicates distress.
